(19) United States
(12) Patent Application Publication
(10) Pub. No.: US 2005/0027183 A1
(43) Pub. Date: Feb. 3, 2005

(54) Method for non-invasive monitoring of blood and tissue glucose

(76) Inventor: Antonio Sastre, Overland Park, KS (US)

Correspondence Address: BLACKWELL SANDERS PEPER MARTIN LLP, 720 OLIVE STREET, SUITE 2400, ST. LOUIS, MO, 63101, US

(21) Appl. No.: 10/881,514

(22) Filed: Jun. 30, 2004

Related U.S. Application Data
(60) Provisional application No. 60/481,049, filed on Jul. 1, 2003.

(51) Int. Cl. A61B 5/00

(52) U.S. Cl. 600/365

(57) ABSTRACT

The present invention provides a non-invasive real-time physiologic measure of blood and tissue glucose wherein measurements of heart rate variability are utilized to track changes in blood and tissue glucose.

### Cross Reference To Related Applications

This application claims the benefit of U.S. Provisional Application Ser. No. 60/481,049 filed Jul. 1, 2003.

### BACKGROUND OF INVENTION

This invention relates generally to a method of determining blood and tissue glucose levels in a mammal, including humans, and, more particularly, to a non-invasive method of determining blood and tissue glucose levels.

Monitoring blood glucose levels is indispensable for the successful control of diabetes. Type I diabetics may need to monitor their blood glucose up to ten times per day, Type II diabetics two to three times per day. Further, blood-glucose levels can serve as an indicator of nutritional, metabolic and health status in non-diabetic individuals. Blood-glucose levels are generally measured by invasive means. For example, home blood-glucose monitoring is generally accomplished by performing a finger-stick with a lancet or other device. A drop of blood is then placed upon a reagent test strip that is analyzed by a glucose monitor. Laboratory blood glucose monitoring is done by drawing a sample of a patient's blood using a needle and directly measuring the glucose therein.

There are currently a small number of physical methods available for non-invasive blood glucose monitoring. These include Near Infrared (NIR) Spectroscopy, NIR Raman Scattering, Kromoscopic™, and Magnetic Resonance. Each of the known methods has serious limitations. An NIR signal, for example, is based upon the interaction of light with all skin layers, subcutaneous tissues, interstitial fluid, and blood. Moreover, each tissue component may have different optical properties and levels of interferents (such as water, fat, protein and hemoglobin), as well as different concentrations of glucose. Hence, NIR can provide only an all-tissue average glucose value, not a specific blood glucose value.

NIR Raman Scattering uses an external light source with a wavelength just above the visible spectrum, then measures the spectrum of scattered radiation. Coupling NIR with NIR Raman Scattering has been used to eliminate skin and tissue effects, thereby enabling a device to detect only blood and fluid glucose. Though this method has the advantages of less interference from water, and sharper bands with less overlap than IR methods, it remains impractical because of the difficulty in removing fluorescence background and tissue-scattering effects.

The proprietary Kromoscopic™ technique is an analog of human color perception wherein photoreceptors for only three colors are able to distinguish thousands of colors. In the case of blood glucose monitoring, a broadband light source and multiple broadband, spectrally overlapping detectors and mathematical transforms and neural nets are used to reconstruct a unique signature for glucose from the responses of the detectors. Though this technique is promising, no human trials have appeared at this time. Thus, whether the approach will be entirely effective is unknown at this time.

Magnetic Resonance devices have cost and size limitations.

What is needed, therefore, is a physiologically based, real-time method of determining blood glucose levels. The present invention provides such a method by correlating heart-rate variability (HRV) to changes in blood glucose levels. Methods for determining and analyzing HRV by conventional time-domain or autoregressive or Fourier spectral methods are known in the art, and International recommendations for uniform usage were issued by a Task Force. Non-conventional methods have been proposed by others. For example, Mallat and colleagues introduced time-series analysis using a wavelet transform modulus mamixa (WTMM) method. Struzik has applied the method of Mallat et al. to constructing the time series of local Holder exponents. Struzik has also provided techniques for the direct measurement of the Holder exponent h(x) of the time series.

Struzik used data from the Beth Israel-MIT cardiac database to show the response of HRV to variations in daily habits and medications. Struzik observed a serendipitious concurrence of changes in h(x) with meal times, but whether this change in HRV was due to blood glucose levels, however, was unknown at the time of the Struzik study.

### REFERENCES

Mallat S, Zhong S. Wavelet transform maxima and multiscale edges. In Wavelets and their applications (eds. Ruskai M. B. et al.) pp. 67-104. Jones and Bartlett Publishers, Boston, 1991.
Mallat S, W L Hwang. Singularity detection and processing with wavelets. IEEE Transactions on Information Theory, 38(2): 617-643, 1992.
Malik M, Camm AJ. Heart rate variability. Futura Publishing Co.: Armonk, N.Y., 1995.
Muzy J F, E Bacry, A Arneodo. The Multifractal Formalism Revisited with Wavelets, International Journal of Bifurcation and Chaos 4:245-302 1994.

Singh J P, Larson MG, O'Donnell C J, et al. Association of hyperglycemia with reduced heart rate variability (The Framingham Heart Study). Am J. Cardiol 2000, 86(3): 309-12
Struzik ZR. Direct Multifractal Spectrum Calculation from the Wavelet Transform. Centrum voor Wiskunde en Informatica Rapport/Informations Systems INS-R9914, Oct. 31, 1999.
Struzik Z R. Revealing Local Variability Properties of Human Heartbeat Intervals with the Local Effective Holder Exponent. Centrum voor Wiskunde Informatica Rapport/Information Systems INS-R0015 Amsterdam, Jun. 30, 2000.
Task Force. Heart rate variability: Standards of measurement, physiological interpretation and clinical use. Special report, Task Force of the European Society of Cardiology and the North American Society of Pacing and Electrophysiology. Circulation 1996;93:1043-1065.

### SUMMARY OF INVENTION

The present invention provides a non-invasive real-time physiologic measure of blood and tissue glucose. It is well known that the autonomic nervous system (ANS) mediates the cephalic (pre-food) insulin response, and that this ANS activity can be detected non-invasively by conventional heart-rate variability (HRV). Preliminary data also demonstrates that specific non-linear, wavelet-derived multifractal characterizations of the HRV signal correlate with meal intakes. The present invention uses this characteristic of HRV to provide a basis for the correlation of HRV and insulin release and glucose levels.

HRV is analyzed by the wavelet transform modulus maxima (WTMM) method as applied to constructing the time series of local Holder exponents. Local Holder exponents are local indices of chaotic and multifractal behavior. These unconventional indices of HRV are those that correlate or precede glycemic changes. Thus, the present invention utilizes these aspects of HRV to monitor blood and tissue glucose levels non-invasively.

### BRIEF DESCRIPTION OF DRAWINGS

FIG. 1 is a graph showing the heart rate variability (HRV) of a healthy adult, plotted as interbeat intervals (IBIs) over time.

FIG. 2 is a graph, taken from the work of Struzik, showing the response of HRV as measured by h(x) to food intake as well as its insensitivity to placebo or a beta-blocker.

### DETAILED DESCRIPTION

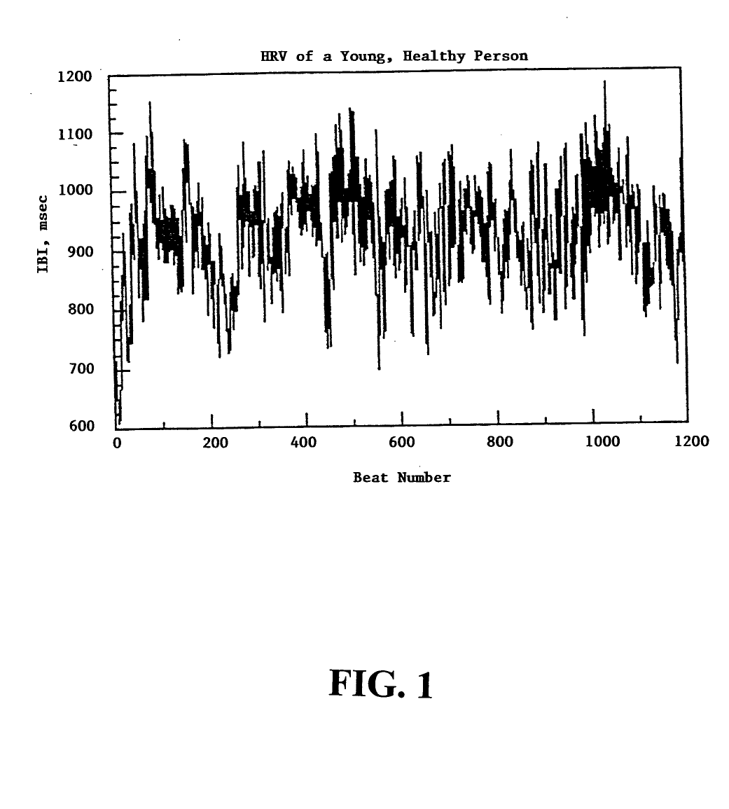

A healthy heart rate is not metronomic. Heart rate variability (HRV) is defined as the variability in cardiac interbeat interval. FIG. 1 provides an illustration of the HRV of a young, healthy person plotted as interbeat interval (IBI) in milliseconds, over time.

HRV has long been recognized as a useful, non-invasive indicator of autonomic nervous system responsiveness, which reflects homeostasis of a number of physiologic variables. Not all of these variables are cardiac in nature. Measurement of HRV can, therefore, provide a convenient, non-invasive window into non-cardiac processes. HRV results from the rhythmic variations in sympathetic and parasympathetic tone to the sino-atrial node of the heart. Specifically, HRV results from the changes in phasic nerve firing in both the sympathetic and parasympathetic branches of the ANS. As both branches of the ANS cope with external stimuli and internal homeostasis (e.g., thermoregulation, blood pressure (BP) control, breathing), their time-varying activity is reflected in HRV. This variability is not conscious, is not experimental noise, and does not reflect somatic reactivity to the external environment. An alternative way to visualize HRV is to note that fluctuations between successive beats are clearly visible as fluctuations above and below the mean heart rate (MHR).

It is a classic, textbook observation that the ANS mediates the cephalic (food-anticipating) release of insulin, and the anatomic and neurotransmitter details of this response at the periphery have been identified. Experimental data using the hyperinsulinimic clamp technique indicates that normal (insulin-responsive) volunteers have an HRV response shifted toward sympathetic dominance upon insulin infusion when compared to non-diabetic but insulin-resistant volunteers, and that the difference is independent of body weight. Similar results on HRV and insulin levels have been obtained using the same techniques on non-diabetic offspring of Type II diabetics. In addition, healthy (Air Force Fighter Pilot trainees), non-diabetic offspring of Type II diabetics exhibit altered HRV when compared with age-matched and lifestyle-matched members of the same Academy. Finally, epidemiologically, hyperglycemia has been associated with reduced HRV in non-diabetic but insuling-resistant individuals.

One way in which HRV is quantified is by first converting a sequence of interbeat intervals (IBI) into a sequence of instantaneous heart rate (IHR) values, typically at a one-second resolution. Spectral or autoregressive analyses of HR sequences (which can encompass from one minute to twenty-four hours) reveal strong periodicities, or bands, ranging from one every two seconds (0.5 Hz) to one every twenty-five seconds (0.04 Hz). It has been established that certain bands are associated with specific autonomic and central nervous system events. For example, the high-frequency (HF) band from 0.15 to 0.4 Hz predominantly reflects the activity of the parasympathetic nervous system as it mediates the respiratory sinus arrhythmia with each inspiration and expiration. Similarly, the weak very-low frequency (VLF) band (<0.04 Hz) and the low-frequency (LF) band (0.04 to 0.15 Hz) have both been associated predominantly with the activity of the sympathetic nervous system mediating efferent thermo-regulatory reflexes, Alternate ways to quantify HRV is to work with the IBI and compute a number of time-domain measures as described by the Task Force. Non-conventional ways of quantification using Holder exponents are described below.

The present invention is based on the discovery that changes in HRV can precede and/or follow insulin release due to reflection of the ANS activity that regulates the neural component of insulin release in some measure of HRV, and that changes in HRV can follow glucose levels due to the participation of neural glucose sensors (e.g. in the medullary area postrema) in the neural component of insulin release, resulting in altered ANS activity reflected in HRV. From what is known of the central and peripheral neuroanatomy, one or both of the above could prove to be the key to tracking blood glucose levels by monitoring HRV. Alterations in HRV can be clearly related to either changes in glucose or to futile attempts by the ANS to provoke the release of insulin from non-existent beta-cells by using simultaneous glucose and HRV measurements of both normal and Type I diabetic volunteers. Preliminary evidence, below, shows that non-conventional ways of quantifying HRV using Holder exponents appear to track meal ingestions.

Determining whether an HRV-derived metric tracks glucose levels in humans can be accomplished by studying normal and Type I diabetic volunteers with simultaneous measurements of HRV and tissue glucose. The Type I diabetic volunteers should be within five years of diagnosis so that the data is not confounded by factors arising from diabetic-induced autonomic neuropathies. Use of Type I diabetic volunteers insures collection of data with significant swings of tissue glucose levels upon meal ingestion and, hence, a large signal for correlation with HRV measures. Volunteers should be studied over a 48-hour period with continuous recording of the electrocardiogram for HRV (using Holter monitors, for example) and continuous tissue glucose monitoring (using, for example, the MiniMed Continuous Glucose Measuring System® (“CGMS”)). The volunteers should track the time, composition and size of meals, and should double-check the calibration and performance of the CGMS with ten finger-sticks per day. Holter data and CGMS data can then be downloaded from the memory units. The EKG can be processed by first identifying the peaks of the R-waves using computer analysis, and from this the series of IBIs obtained. CGMS data can be used as raw data (tissue glucose levels v. time).

Examination of cardiac IBI (shown in FIG. 1) demonstrates the complexity in the signal, which contains the periodic components (e.g. LF, HF) detected by the conventional spectral measures, as well as chaotic components. A large body of research suggests that IBIs share many characteristics with complex non-linear systems. Thus, IBIs can be usefully analyzed using techniques initially developed for statistical physics of chaotic dynamical systems.

The ‘roughness’ of the IBI graph can be analyzed using techniques that rely on wavelet transforms (WT). A WT is simply the convolution of a signal f(x) with special functions (“wavelets”) that permit localization in time, subject to translations and dilations, and obeying finiteness and orthogonality conditions. The roughness of f(x) is quantified by noting how quickly the time series jumps from one value to the next. We examine those points x (in time) for which the change f(x+1)−f(x) can be written as O(l^{h(x)}). The exponent h(x) is a Holder exponent; the smaller h(x) is, the rougher the signal appears near x, whereas large h(x) values are characteristic of smooth curves. While direct numerical calculation of such exponents is unstable, it has been observed by Mallat et al. that the WT can be used to simultaneously detrend data and to extract Holder exponents via the WT Modulus Maximum (WTMM). Let Ψ denote an analyzing wavelet with an appropriate number of vanishing moments. About each point x in the time series, we can form the cone |y−x|/a<C in the (y, a) half-plane over which the continuous wavelet transform is defined. For 0<h<1, the Holder exponent h(x) may then be computed at the largest exponent h satisfying the condition:

$${max}_{\vert y - x \vert \leq Ca} \vert {W}_{ψ} f ( y , a ) \vert = O ( {a}^{h} )$$

where W_{Ψ}f (y, a) is the wavelet transform evaluated at time y and scale a,

$${W}_{ψ} f ( y , a ) = \frac{1}{a} \int ψ ( \frac{y - x}{a} ) f ( x ) ⅆ x$$

It has been observed by Muzy et al. that these smoothed transforms of the data allow accurate statistics on h(x) to be computed. The statistic computed, called the multifractal spectrum, measures the fractal dimension of the collection of x data points that share a common Holder exponent h(x). The shape of the fractal spectrum reveals how deeply intertwined roughness is within IBI; from it, one may also compute correlations. It has been shown to be stable and accurate in theoretical studies.

This spectrum, however, as a statistic that averages over the entire time-series of the IBI, cannot localize short-time events such as are important for the present method. It is well known that the WTMM method analyzes curves with highly irregular shapes to produce limiting sequences for which the slope is difficult to determine. This makes it difficult to examine h(x)versus x. Techniques for direct measurement of h(x) have also been demonstrated. One such technique, by Struzik, takes advantage of the fact that the wavelet transform of a multiplicative process and its derivations from linearity are well understood. The variations in the wavelet transforms of IBIs are modeled as deriving from a multiplicative process, which allows accurate fitting of the data. From this, the local Holder exponent series, h(x), is derived.

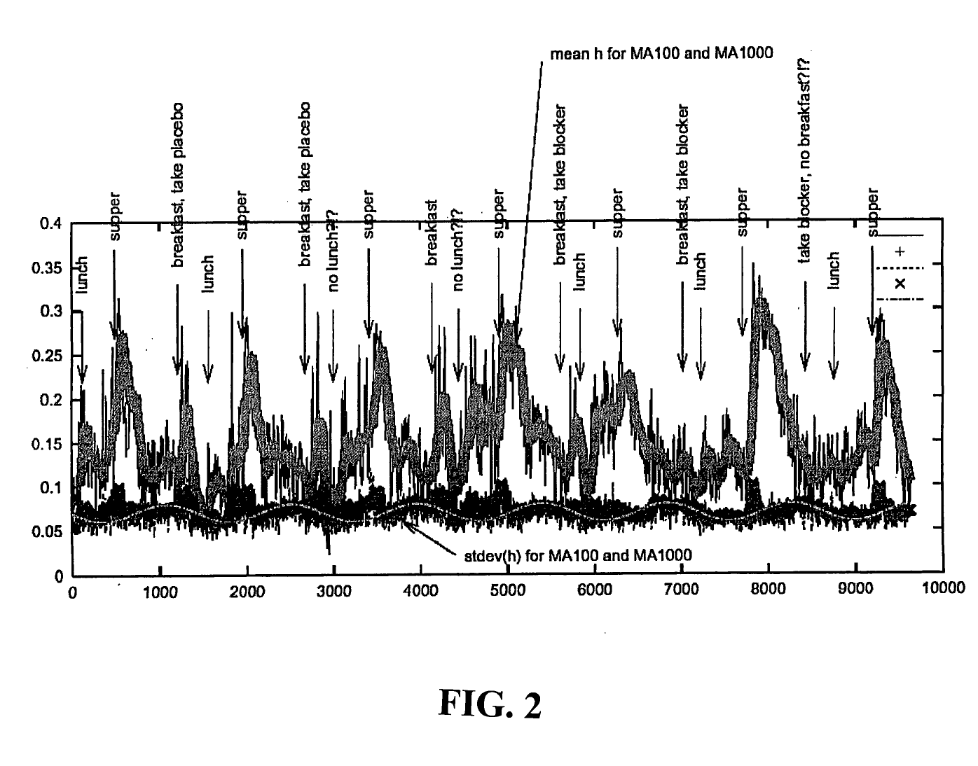

Once the local Holder exponent is computed, an accurate comparison of a broad range of statistics is possible. Even the raw time series of h(x) values correlate with events of physiologic importance. Data from the Beth Israel-MIT cardiac database show a remarkable response of HRV to food intake, as well as the remarkable insensitivity of the h(x) series (vertical axis) to the patient taking a placebo or a beta-blocker (see FIG. 2, from Struzik). The beat number is the horizontal axis in FIG. 2. This is in marked contrast to the conventional, spectral measures that would be very sensitive to the beta-blocker. Whether this response to food intake is due to glycemic events (changes in blood or tissue glucose, or insulin release), or to gut muscle activity cannot be ascertained from the data previously used, but these results show the complementary nature of the information that can be derived from spectral and non-linear dynamics-derived measures applied to HRV. These results also show that HRV is altered by food ingestion.

There are a number of possible variations on the computations of time series of Holder exponents h(x) which remain to be explored. In addition, it is well known that the glucose tissue levels measured by the MiniMed® CGMS will lag or lead blood levels depending on whether blood levels are rising or falling. This is not an artifact of the measuring device but an inescapable consequence of the tissue compartments through which glucose is delivered and metabolized. These lags and leads will be examined when attempting to correlate the time series h(x) with the CGMS values.

Thus, the present invention provides a novel method of obtaining accurate blood glucose levels from entirely non-invasive physiologic means. By measuring HRV and correlating changes in HRV with changes in glucose levels, one is able to carefully and, if necessary, continuously monitor the blood glucose levels of an individual, thereby providing superior control and management of disease states such as diabetes.

In addition to monitoring blood glucose levels to control diabetes in diabetic individuals, the present invention may also be used in conjunction with telemetry devices to remotely monitor blood glucose levels of individuals. This is important because, in a non-diabetic individual, blood glucose levels can be an indicator of overall health status, as well as an individual's metabolic and nutritional state.

It is understood that the description and examples above are exemplary in nature and are not intended to be limiting. Changes and modifications may be apparent to one skilled in the art upon reading this disclosure, and such changes and modifications may be made without departing from the spirit and scope of the present invention.

1. A non-invasive physiologic method for determining blood glucose levels comprising:
computing non-conventional measures of the heart rate variability of an individual over time; and
correlating said heart rate variability with the individual's blood glucose level.

2. A non-invasive physiologic method for determining blood glucose levels comprising:
(a) measuring the cardiac interbeat interval of an individual;
(b) performing a wavelet transform of the interbeat interval data measured in step (a) above;
(c) extracting the Holder exponents from the wavelet transformed interbeat interval data measured in step (a) above; and
(d) correlating the raw time series of said Holder exponent with the individual's blood glucose level.
